(19) United States
(12) Patent Application Publication
(10) Pub. No.: US 2016/0360972 A1
(43) Pub. Date: Dec. 15, 2016

(54) BIOLOGICAL INFORMATION MEASUREMENT DEVICE, DEVICE PROVIDED WITH SAME, AND BIOLOGICAL INFORMATION MEASUREMENT SYSTEM

(71) Applicant: PANASONIC INTELLECTUAL PROPERTY MANAGEMENT CO., LTD., Osaka-shi (JP)

(72) Inventors: Tomoya KUSAKABE, Shiga (JP); Kazuhiro IDE, Osaka (JP)

(21) Appl. No.: 15/120,984

(22) PCT Filed: May 12, 2015
(86) PCT No.: PCT/JP2015/002393

§ 371 Date: Aug. 23, 2016

(30) Foreign Application Priority Data
May 13, 2014 (JP) 2014-099397

(51) Int. Cl. A61B 5/0205; A61B 5/00; A61B 5/11

(52) U.S. Cl. CPC A61B 5/0205; A61B 5/1118; A61B 5/6824; A61B 5/4041; A61B 5/681; A61B 5/02416

(57) ABSTRACT

A biological information measurement device includes a pulse wave detector having a light emitter for irradiating a living body with light and a light receiver for receiving reflected light of the light emitted from the living body when the light emitter irradiates the living body with the light, and detecting pulse wave information of the living body based on the reflected light received in the light receiver; an operator for determining an autonomic nerve function based on the pulse wave information of the living body detected by the pulse wave detector; and a controller configured so as to control the pulse wave detector and the operator. The pulse wave detector, the operator, and the controller are provided in a measuring device main body case.

### TECHNICAL FIELD

The invention relates to a biological information measurement device and a biological information measurement system having the device.

### BACKGROUND ART

In recent years, the balance between a function of a sympathetic nerve and a function of a parasympathetic nerve has not been able to be obtained due to the increase of various mental and physical stresses according to social environment and living environment. Therefore, a problem of deterioration in an autonomic nerve function occurs.

Devices for evaluating the presence or absence of the deterioration in the autonomic nerve function have been developed. As an example of a device for evaluating deterioration of the autonomic nerve function, an autonomic nerve function evaluation device for displaying, where a function of a sympathetic nerve and a parasympathetic nerve of a user is in any one of enhancement, normal, and deterioration based on data which is obtained by measuring the heart sound of the user in a 2D map, is known (for example, refer to PTL 1).

In the conventional autonomic nerve function evaluation device, for measuring the heart sound of the user, a negative electrode is to be mounted on the user's throat, a positive electrode is to be mounted on the user's left side, and a ground electrode is to be mounted on the user's right side. In addition, the conventional autonomic nerve function evaluating device is electrically connected to each electrode and has an electrocardiogram monitor for acquiring heart sound data, a control device for evaluating activities of a sympathetic nerve and a parasympathetic nerve based on the heart sound data, and a display device for displaying an evaluating result.

According to the conventional autonomic nerve function evaluation device, the electrocardiogram monitor, the control device, and the display device are formed separate from each electrode. Therefore, when the user moves in a state where each electrode is mounted on the user, the user is required to grasp the electrocardiogram monitor, the control device, and the display device. Therefore, the user cannot carry the conventional autonomic nerve function evaluation device.

### CITATION LIST

### Patent Literature

PTL 1: Japanese Patent Unexamined Publication No. 2010-172365

### SUMMARY OF THE INVENTION

The invention provides a biological information measurement device and a biological information measurement system having the same which can be carried by a user.

The biological information measurement device of the invention includes a pulse wave detector having a light emitter for irradiating a living body with light and a light receiver for receiving reflected light of the light emitted from the living body when the light emitter irradiates the living body with the light, and detecting pulse wave information of the living body based on the reflected light received in the light receiver. The biological information measurement device includes an operator for determining an autonomic nerve function based on the pulse wave information of the living body detected by the pulse wave detector; a controller configured so as to control the pulse wave detector and the operator; and a measuring device main body case provided with the pulse wave detector, the operator, and the controller.

According to the configuration, the biological information measurement device, in which a device main body includes a pulse wave detector for detecting a pulse wave by an optical system, determines an autonomic nerve function. Therefore, the electrocardiogram monitor for detecting the pulse wave and a control device for determining the autonomic nerve function are not formed separate from the device main body. Accordingly, the configuration which allows the user to carry the biological information measurement device can be obtained.

### BRIEF DESCRIPTION OF DRAWINGS

FIG. 1A is a front view of a biological information measurement device according to a first exemplary embodiment of the invention.

FIG. 1B is a rear view of a biological information measurement device according to the first exemplary embodiment of the invention.

FIG. 1C is a front view of the biological information measurement device in a state where the device is mounted on a wrist, in the first exemplary embodiment of the invention.

FIG. 2 is a block diagram illustrating a controlling configuration of the biological information measurement device of the first exemplary embodiment of the invention.

FIG. 3A is a diagram illustrating a degree of autonomic nerve activity of the first exemplary embodiment of the invention.

FIG. 3B is a diagram illustrating the number of steps according to the first exemplary embodiment of the invention.

FIG. 3C is a diagram illustrating a sleeping time of the first exemplary embodiment of the invention.

FIG. 4A is a scattering diagram illustrating a relationship between the degree of the autonomic nerve activity and the number of steps of the first exemplary embodiment of the invention.

FIG. 4B is a scattering diagram illustrating the relationship between the degree of the autonomic nerve activity and the sleeping time of the first exemplary embodiment of the invention.

FIG. 5 is a flow chart illustrating a procedure of a determination of an autonomic nerve function of the first exemplary embodiment of the invention.

FIG. 6 is a flow chart illustrating the procedure of a measurement of the sleeping time of the first exemplary embodiment of the invention.

FIG. 7 is a flow chart illustrating a procedure of a determination of an autonomic nerve function of a second exemplary embodiment of the invention.

FIG. 8A is a diagram illustrating a body motion signal of the second exemplary embodiment of the invention.

FIG. 8B is a diagram illustrating a light emitting state of a light emitter of the second exemplary embodiment of the invention.

FIG. 8C is a diagram illustrating a light receiving signal of the second exemplary embodiment of the invention.

FIG. 9 is a diagram illustrating a relationship between an On DUTY ratio and a body motion signal of a driving circuit of the light emitter of a third exemplary embodiment of the invention.

FIG. 10 is a block diagram illustrating a control configuration of a biological information measurement device of a fourth exemplary embodiment of the invention.

FIG. 11 is a diagram illustrating a relationship between a gain and a body motion signal of a light receiver of the fourth exemplary embodiment of the invention.

FIG. 12 is a configuration diagram illustrating a configuration of a biological information measurement device of a fifth exemplary embodiment of the invention.

FIG. 13 is a block diagram illustrating a control configuration of a biological information measurement system of a sixth exemplary embodiment of the invention.

FIG. 14 is a radar chart illustrating a comprehensive evaluation which is obtained by using the biological information measurement device of a modification example of the invention.

### DESCRIPTION OF EMBODIMENTS

Hereinafter, an exemplary embodiment of the invention will be described with reference the drawings.

### First Exemplary Embodiment

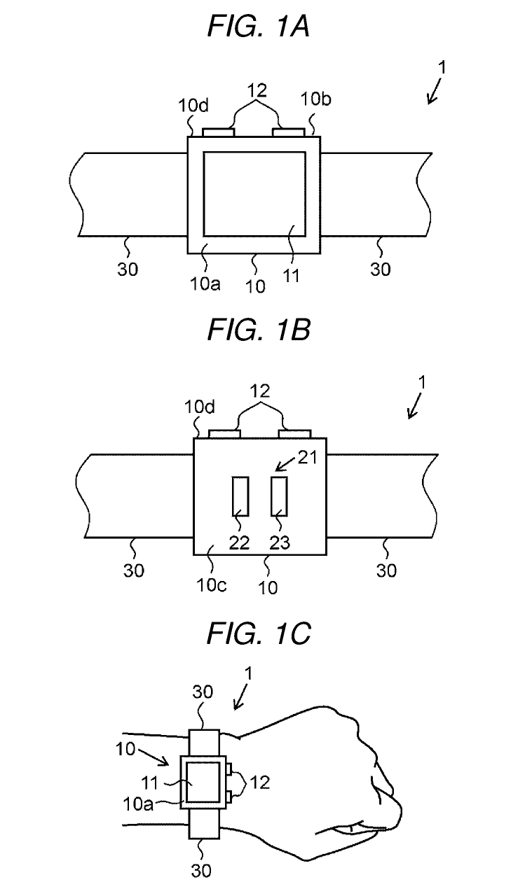

FIG. 1A is a front view of biological information measurement device 1 of a first exemplary embodiment of the invention. FIG. 1B is a rear view of biological information measurement device 1 shown in FIG. 1A. FIG. 1C is a front view of biological information measurement device 1 in a state where the device is mounted on a wrist.

As shown in FIG. 1A, biological information measurement device 1 includes device main body 10 having device main body case 10a, and a pair of mounts 30 which is attached to both sides of device main body case 10a. An example of mount 30 is a belt. Display 11 formed of a liquid crystal display is provided on surface 10b of device main body case 10a. Input component 12 formed of an operation button is provided on side surface 10d of device main body case 10a. In addition, as shown in FIG. 1B, light emitter 22 and light receiver 23, which are included in pulse wave detector 21 for measuring a pulse wave of a user, are provided on rear surface 10c of a case of device main body 10. Light emitter 22 and light receiver 23 are adjacent to each other.

As shown in FIG. 1C, biological information measurement device 1 is mounted on a wrist of the user that is a living body by mount 30. In this state, light emitter 22 and light receiver 23 of pulse wave detector 21 in FIG. 1B are disposed so as to face the wrist.

Here, a detection principle of a pulse wave by pulse wave detector 21 will be described. In a case where light emitter 22 radiates light to the wrist, an amount of reflected light reflected from the irradiated wrist is changed due to changes in an amount of hemoglobin associated with a heartbeat. Light receiver 23 receives the reflected light and generates a light receiving signal in accordance with the amount of the reflected light. Since the amount of the reflected light (hereinafter, referred to as an “amount of received light”) to be received in light receiver 23 is changed by a pulsation of a blood associated with the heartbeat, the light receiving signal is changed according to a cycle of the pulsation. Therefore, pulse wave detector 21 can detect the pulse wave based on changes in the light receiving signal.

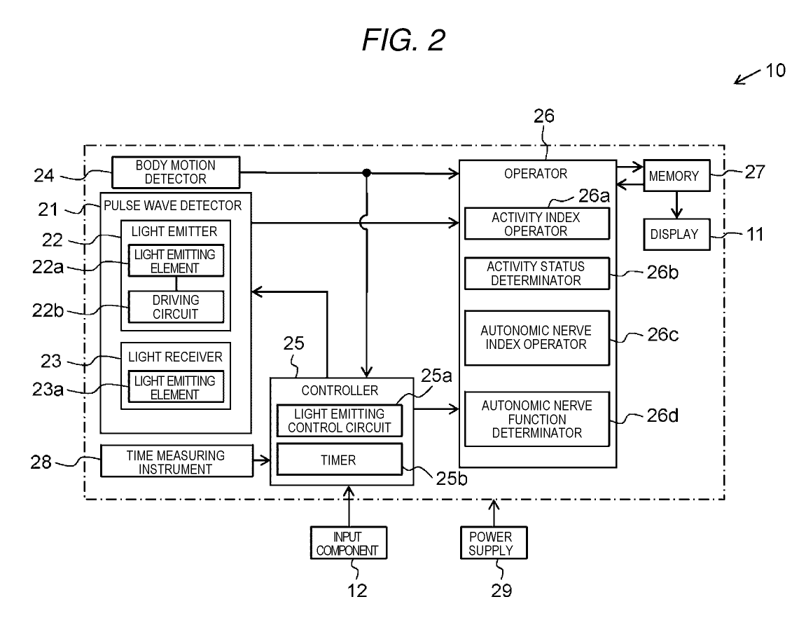

FIG. 2 is a block diagram illustrating a controlling configuration of biological information measurement device 1 of the first exemplary embodiment of the invention.

As shown in FIG. 2, device main body 10 includes display 11, input component 12, pulse wave detector 21, body motion detector 24, controller 25, operator 26, memory 27, time measuring instrument 28, and power supply 29. Device main body 10 measures the number of steps and a sleeping time, determines the deterioration in an autonomic nerve function and determines a cause of the autonomic nerve function deterioration (hereinafter, referred to as a determination of the autonomic nerve function) based on the pulse wave, the number of steps, and the sleeping time.

Pulse wave detector 21 includes light emitter 22 and light receiver 23. Light emitter 22 includes light emitting element 22a and driving circuit 22b. An example of light emitting element 22a is a red light emitting diode. Driving circuit 22b is configured to switch between supply and cut-off of power to light emitting element 22a.

Light receiver 23 includes light receiving element 23a. An example of light receiving element 23a is a photodiode. Light receiver 23 outputs the light receiving signal in accordance with the amount of the received light to operator 26.

Body motion detector 24 is provided with an acceleration sensor. Body motion detector 24 detects the body motion of the living body as an acceleration of the living body. Furthermore, body motion detector 24 outputs the body motion signal in accordance with the acceleration to controller 25 and operator 26.

Controller 25 controls pulse wave detector 21, body motion detector 24, and operator 26. In addition, controller 25 controls all or some of display 11, input component 12, memory 27, time measuring instrument 28, and power supply 29.

Controller 25 includes light emitting control circuit 25a for controlling driving circuit 22b of light emitter 22 and timer 25b for measuring the time. Controller 25 obtains the body motion signal which is detected by body motion detector 24 in a predetermined sampling cycle. Controller 25 includes a measuring mode performing a determination of the autonomic nerve function, a sleeping mode measuring a sleeping time, and a standby mode without determining of the autonomic nerve function and measuring of the sleeping time. Controller 25 is configured to switch between each of the control modes based on the operation of input component 12.

When controller 25 is in the measuring mode, the detection of the pulse wave is performed by pulse wave detector 21 and when controller 25 is in the sleeping mode, the detection of the pulse wave is not performed by pulse wave detector 21. On the other hand, the measurement of the number of steps is performed regardless of each control mode of controller 25. Therefore, the detection of the acceleration is performed by body motion detector 24 regardless of each control mode of controller 25.

Operator 26 includes activity index operator 26a, activity status determinator 26b, autonomic nerve index operator 26c, and autonomic nerve function determinator 26d. When controller 25 is in the measuring mode, operator 26 obtains the light receiving signal of pulse wave detector 21 in the predetermined sampling cycle. Operator 26 obtains the body motion signal from body motion detector 24 in the predetermined sampling cycle regardless of the control modes of controller 25.

Activity index operator 26a calculates the number of steps that is an example of an activity status and the sleeping time as an activity index based on the body motion signal obtained from body motion detector 24.

Activity status determinator 26b determines whether the body motion signal obtained from body motion detector 24 is equal to or more than predetermined threshold value Tr (first predetermined threshold value). Threshold value Tr is a value for discriminating what the user is in a motion such as walking and is set by an examination or the like in advance.

Autonomic nerve index operator 26c calculates a degree of the autonomic nerve activity that is an autonomic nerve index. An example of the degree of the autonomic nerve activity is entropy value E of the heartbeat variability. The method of calculating entropy value E is as follows.

Autonomic nerve index operator 26c calculators a cardiac cycle that is one course of the contraction and relaxation of the heart (hereinafter, referred to as “heart beat interval PP”) at first. Heart beat interval PP is an example of pulse wave information detected by pulse wave detector 21, and measured as a time between a maximum value of the light receiving signal and the maximum value of the next light receiving signal. Next, autonomic nerve index operator 26c calculates a percentage index (PI) value indicating by percentage the changes in heart beat interval PP. The PI value is calculated by the following (Formula 1) expression.

$$PI ( n ) = \frac{{PP}_{i} - {PP}_{i + 1}}{{PP}_{i}} \times 100$$

Autonomic nerve index operator 26c calculates entropy value E based on the following expression (Formula 2), based on a probability distribution of the PI value calculated by the (Formula 1) expression. p(i) in the (Formula 2) expression represents the probability distribution of the PI value.

$$E = {\sum }_{i = 1}^{n} p ( i ) {log}_{2} p ( i )$$

As shown in the (Formula 2) expression, since two or more N's p(i)'s are used, data of N heart beat intervals PP is required.

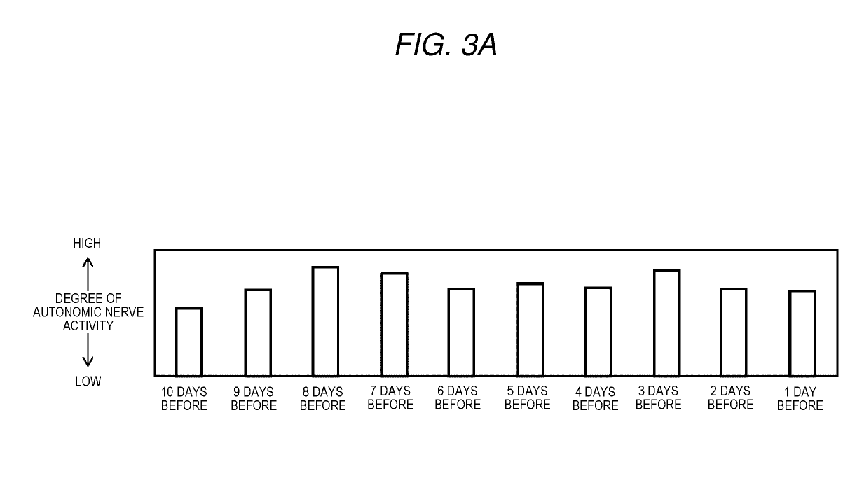

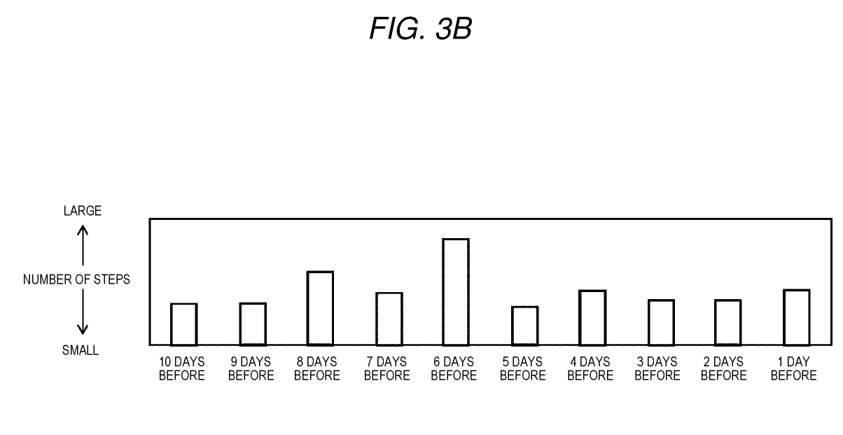

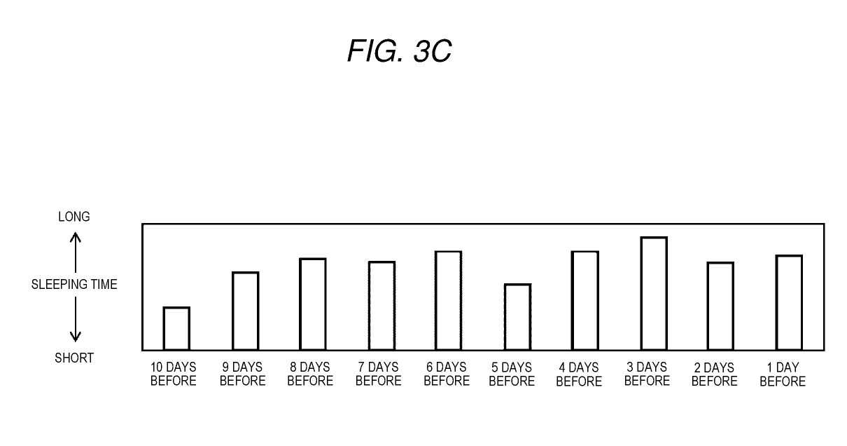

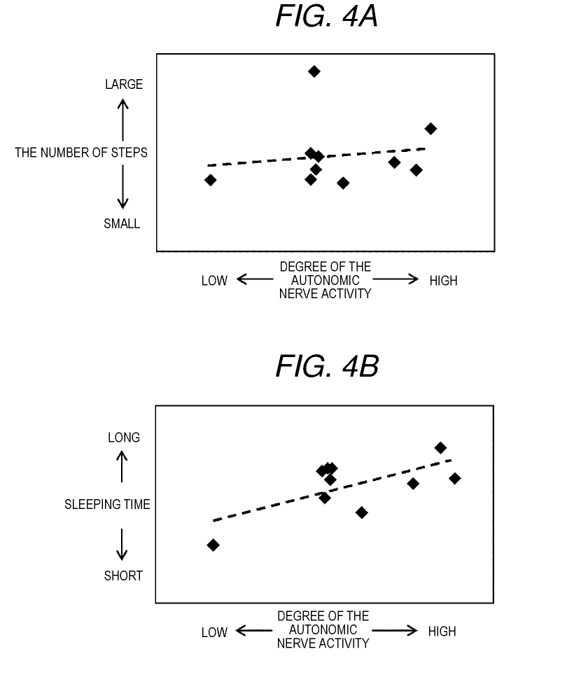

FIG. 3A is a diagram illustrating a degree of autonomic nerve activity of the first exemplary embodiment of the invention. FIG. 3B is a diagram illustrating the number of steps according to the first exemplary embodiment of the same. FIG. 3C is a diagram illustrating a sleeping time of the first exemplary embodiment of the same. FIG. 4A is a scattering diagram illustrating a relationship between the degree of the autonomic nerve activity and the number of steps of the first exemplary embodiment of the invention. FIG. 4B is a scattering diagram illustrating the relationship between the degree of the autonomic nerve activity and the sleeping time of the first exemplary embodiment of the same.

In FIGS. 3A to 3C, then numbers 1 (day before) to 10 (days before) indicates how many days before data from the present time. For example, “10 days before” in FIG. 3A indicates the degree of the autonomic nerve activity 10 days before from the present time.

As an example, autonomic nerve function determinator 26d calculates correlation coefficient between the degree of the autonomic nerve activity and the number of steps using the measurement result of the degree of the autonomic nerve activity and the measurement result of the number of steps as shown in FIGS. 3A and 3B, respectively. In addition, as shown in FIG. 4A, autonomic nerve function determinator 26d generates a scattering diagram of the relationship between the degree of the autonomic nerve activity and the number of steps. In addition, as an example, autonomic nerve function determinator 26d calculates the correlation coefficient between the degree of the autonomic nerve activity and the sleeping time using the measurement result of the degree of the autonomic nerve activity and the measurement result of the sleeping time as shown in FIGS. 3A and 3C, respectively. In addition, as shown in FIG. 4B, autonomic nerve function determinator 26d generates the scattering diagram of the relationship between the degree of the autonomic nerve activity and the sleeping time.

In addition, autonomic nerve function determinator 26d determines whether the autonomic nerve function is deteriorated or not based on whether entropy value E is equal to or more than threshold value Tk (second predetermined threshold value). When the degree of the autonomic nerve activity is less than threshold value Tk, autonomic nerve function determinator 26d determines that the autonomic nerve function is deteriorated. On the other hand, when the degree of the autonomic nerve activity is equal to or more than threshold value Tk, autonomic nerve function determinator 26d determines that the autonomic nerve function is not deteriorated.

Threshold value Tk indicates a minimum value of the range of the degree of the autonomic nerve activity in which the autonomic nerve function becomes in a normal state, and is a value for discriminating what the autonomic nerve function is deteriorated. Threshold value Tk is set in advance by an examination or the like.

When it is determined that the autonomic nerve function is deteriorated, autonomic nerve function determinator 26d determines that a cause of the deterioration of the autonomic nerve function is any one of the walking and the sleeping time as follows.

Firstly, autonomic nerve function determinator 26d determines whether the correlation coefficient between the number of steps and the degree of the autonomic nerve activity is equal to or more than threshold value Ts (third predetermined threshold value). When the correlation coefficient is equal to or more than threshold value Ts, autonomic nerve function determinator 26d determines that the number of steps is a cause of the deterioration of the autonomic nerve function. Next, autonomic nerve function determinator 26d determines whether a correlative relationship between the sleeping time and the degree of the autonomic nerve activity is equal to or more than threshold value Ts.

When the correlation coefficient is equal to or more than threshold value Ts, autonomic nerve function determinator 26d determines that the sleeping time is a cause of the deterioration of the autonomic nerve function. In addition, threshold value Ts is set in advance by the examination or the like. As an example, as shown in the scattering diagrams of FIGS. 4A and 4B, the sleeping time having a light correlation coefficient between the sleeping time and the autonomic nerve function than that of the number of steps. When the correlation coefficient is equal to or more than threshold value Ts, autonomic nerve function determinator 26d determines that the sleeping time is a cause of the deterioration of the autonomic nerve function.

An example of memory 27 is a random access memory (RAM). Memory 27 stores the degree of the autonomic nerve activity, the number of steps, and the sleeping time for 10 days, for example. When the autonomic nerve activity is measured in several times a day, the degree of the autonomic nerve activity may be stored as an average value of the measurement results. The number of steps is stored as an accumulated value of the number of steps of a day.

In addition, the sleeping time is stored as a sleeping time measured in a sleeping mode in a day. Memory 27 deletes an activity index and an autonomic nerve index dated prior to 11 days before. In addition, threshold value Tr used for the activity status determination, and threshold values Tk and Ts used for the autonomic nerve function determination are stored in memory 27. In addition, information for alleviating the deterioration of the autonomic nerve function (hereinafter, referred to as “advice information”) is stored in memory 27.

Time measuring instrument 28 measures a present time. Time measuring instrument 28 includes a quartz watch as an example. Time measuring instrument 28 outputs the result that the present time is measured as a time signal to controller 25.

Power supply 29 is a secondary battery, and supplies a power to pulse wave detector 21, body motion detector 24, controller 25, operator 26, memory 27, time measuring instrument 28, and display 11.

Display 11 displays a bar graph of the degree of the autonomic nerve activity, the number of steps, and the sleeping time for 10 days shown in FIGS. 3A to 3C, a scattering diagram of the relationship between the degree of the autonomic nerve activity and the number of steps shown in FIGS. 4A and 4B, and a scattering diagram of the relationship between the degree of the autonomic nerve activity and the sleeping time. In addition, display 11 displays the measurement result of the autonomic nerve function and the advice information. By the operation of input component 12, display 11 can display the above-described measurement result of the degree of the autonomic nerve activity, the number of steps, and the sleeping time, a cause of the deterioration of the autonomic nerve function, and the advice information by strolling.

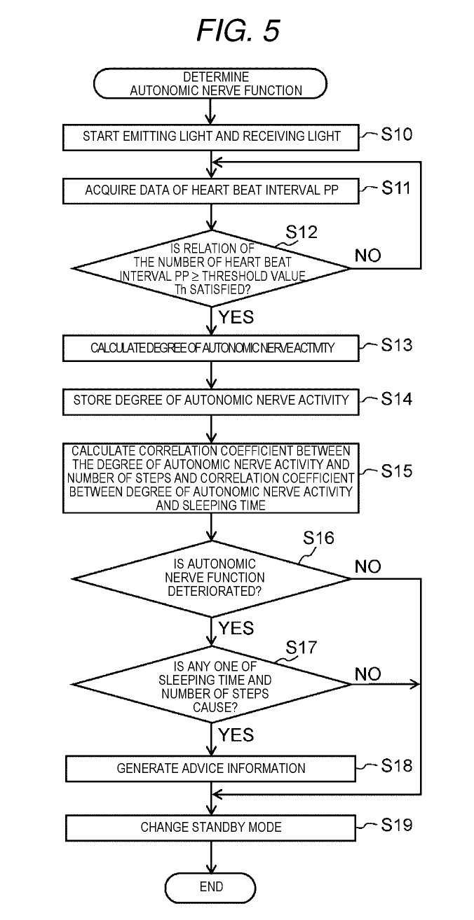

FIG. 5 is a flow chart illustrating a procedure of a determination of an autonomic nerve function of the first exemplary embodiment of the invention.

The determination of the autonomic nerve function to be executed by device main body 10 will be described using FIG. 5 with reference to FIGS. 1 and 2. In the flowing description, a portion of biological information measurement device 1 with a reference numeral indicates a portion of biological information measurement device 1 of FIG. 2.

When the user operates input component 12 to set the measuring mode, the determination of the autonomic nerve function starts.

At first, controller 25 starts light emitting of light emitter 22 and light receiving of light receiver 23 (Step S10). Therefore, light emitting element 22a emits light toward the wrist and light receiving element 23a receives reflected light from the wrist.

Next, autonomic nerve index operator 26c acquires data of heart beat interval PP (Step S11) and determines whether the number of data items of heart beat interval PP is equal to or more than threshold value Th (fourth predetermined threshold value) (Step S12). Threshold value Th is a minimum value of the number of data items of heart beat interval PP which is required for calculating entropy value E and is set in advance by an examination or the like.

When it is determined that the number of data items of heart beat interval PP is not equal to or more than threshold value Th (NO in Step S12), autonomic nerve index operator 26c determines that the data of heart beat interval PP which is required for calculating entropy value E is insufficient, proceeds to Step S11, and acquires data of heart beat interval PP again.

On the other hand, when it is determined that the number of data items of heart beat interval PP is equal to or more than threshold value Th (YES in Step S12), autonomic nerve index operator 26c calculates entropy value E that is the degree of the autonomic nerve activity based on the data of heart beat interval PP (Step S13).

After autonomic nerve index operator 26c calculates the degree of the autonomic nerve activity, memory 27 stores the degree of the autonomic nerve activity (Step S14), and autonomic nerve function determinator 26d calculates the correlation coefficient between the number of steps and the degree of the autonomic nerve activity, and between the sleeping time and the degree of the autonomic nerve activity (Step S15). Autonomic nerve function determinator 26d determines whether the autonomic nerve function is deteriorated (Step S16).

When the autonomic nerve function is deteriorated in Step S16 (Step S16, YES), autonomic nerve function determinator 26d determines whether the cause of the deterioration of the autonomic nerve function is at least one of the number of steps and the sleeping time (Step S17). When it is determined that the cause of the deterioration of the autonomic nerve function is at least one of the number of steps and the sleeping time (YES in Step S17), autonomic nerve index operator 26c generates advice information (Step S18).

The advice information is obtained from memory 27 based on the cause of the deterioration of the autonomic nerve function which is determined in Step S17. In a case where the cause of the deterioration of the autonomic nerve function is the sleeping time, as an example, the advice information that “You are in these 10 days, the degree of the autonomic nerve activity is likely to be affected due the sleeping time. Let's take an enough sleeping time and try not to accumulate the stress.” is obtained from memory 27 and displayed on display 11.

Finally, controller 25 set a standby mode (Step S19). Therefore, light emitting of light emitter 22 of pulse wave detector 21 is interrupted. On the other hand, in any one of Step S16 and Step S17, it is determined NO, the process proceeds to Step S19.

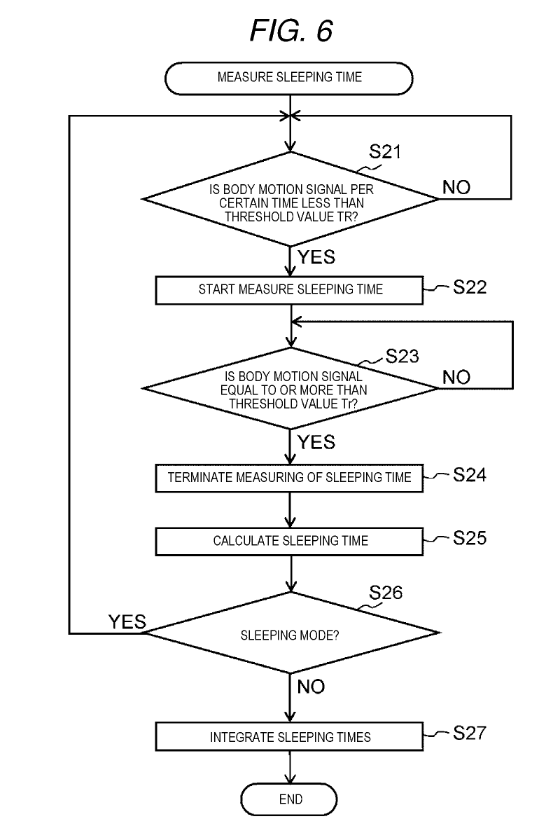

FIG. 6 is a flow chart illustrating the procedure of a measurement of the sleeping time of the first exemplary embodiment of the invention.

The measurement of the sleeping time to be executed by device main body 10 will be described using FIG. 6 with reference to FIGS. 1 and 2. When the user operates input component 12 to set a sleeping mode at bedtime, the measurement of the sleeping time is started. In the flowing description, a portion of biological information measurement device 1 with a reference numeral indicates portions of biological information measurement devices 1 of FIGS. 1 and 2.

Activity status determinator 26b determines whether the body motion signal is less than threshold value Tr during the predetermined time (first predetermined time) (Step S21). The predetermined time means a time for discriminating that the user is in a sleeping status or not, and is set in advance by an examination or the like.

When the body motion signal is less than threshold value Tr (YES in Step S21), activity index operator 26a starts measuring the sleeping time (Step S22). On the other hand, when it is determined that the body motion signal is not less than threshold value Tr (NO in Step S21), the process proceeds to Step S21 again.

After the measurement of the sleeping time is started, activity status determinator 26b determines whether the body motion signal is equal to or more than threshold value Tr (Step S23). When it is determined that the body motion signal is equal to or more than threshold value Tr (YES in Step S23), activity index operator 26a determinates the measuring the sleeping time (Step S24), and calculates the sleeping time (Step S25). On the other hand, when it is determined that the body motion signal is not equal to or more than threshold value Tr in Step S23 (NO in Step S23), the process proceeds to the determination in Step S23 again.

After terminating the calculation of the sleeping time by activity index operator 26a, controller 25 determines whether it is in the sleeping mode (Step S26). By the determination of Step S26, it is determined that the body motion of the user which is determined in Step S23 is in a state where the user gets up temporally, that is, the user falls asleep immediately after getting up. In a case where the user getting up and not fall asleep immediately after getting up, the user operates input component 12 to set the standby mode or the measuring mode from the sleeping mode. On the other hand, in a case where the user gets up and then falls asleep again immediately getting up, the user does not operate input component 12. Therefore, the sleeping mode is maintained.

When it is determined that there is in the sleeping mode (YES in Step S26), the process proceeds to Step S21, and activity index operator 26a executes the measurement of the sleeping time again. On the other hand, when it is determined that where is no sleeping mode (NO in Step S26), activity index operator 26a calculates the calculation result of the sleeping time (Step S27). When the calculation of the sleeping time in Step S25 is performed at only one time and the process proceeds to Step S27, the result which is obtained by integrating the calculating results of the sleeping time is the same as the calculating result of the sleeping time in Step S25. Through such a procedure, the sleeping time between when the user goes to bed and when the user gets up is measured.

Here, the action of biological information measurement device 1 of the exemplary embodiment will be described.

The autonomic nerve function is deteriorated due to an increase in the stress with lack of sleep and lack of activity or an increase in the stress caused by fatigue due to excessive activity.

In device main body 10, when the autonomic nerve function id deteriorated, autonomic nerve function determinator 26d calculates the correlation coefficient between the degree of the autonomic nerve activity and the number of steps and the correlation coefficient between the degree of the autonomic nerve activity and the number of steps. Therefore, the relationship between the autonomic nerve function and the number of steps and the sleeping time can be found. In a case where any one of the number of steps and the sleeping time is closely related to the autonomic nerve function, autonomic nerve function determinator 26d determines that the activity index which is closely related to the autonomic nerve function is a cause of the deterioration of the autonomic nerve function. Therefore, biological information measurement device 1 can perform the determination of a total health status where the autonomic nerve function, the number of steps, and the sleeping time are combined.

That is, in device main body 10, body motion detector 24 detects the body motion of the living body and pulse wave detector 21 detects pulse wave information of the living body. Operator 26 determines the autonomic nerve function based on the detected pulse wave information of the living body and the detected body motion information of the living body.

Accordingly, from the autonomic nerve function and the activity status based on the body motion information of the living body detected by body motion detector 24, the relationship between the autonomic nerve function and the activity status can be found. Therefore, when the autonomic nerve function is deteriorated, it can be determined that the activity having a high relationship between the autonomic nerve function and thereof is a cause of the deterioration of the autonomic nerve function.

In addition, device main body 10 provides information for alleviating the deterioration of the autonomic nerve function based on the determination result of the type of the activity status which causes the deterioration of the autonomic nerve function. For example, when display 11 displays the degree of the autonomic nerve activity, the measurement result of the sleeping time, a cause of the deterioration of the autonomic nerve function, and the advice information, these information items can be provided to the user.

Biological information measurement device 1 of the exemplary embodiment has the flowing effects.

Device main body 10 of biological information measurement device 1 includes pulse wave detector 21 and body motion detector 24 and determines the deterioration of the autonomic nerve function and a cause of the deterioration of the autonomic nerve function. Therefore, the electrocardiogram monitor for detecting the pulse wave and the control device for determining the autonomic nerve function may not be provided separated from the device main body like the conventional autonomic nerve function evaluation device. Accordingly, the user can carry biological information measurement device 1. The user performs the determination of the autonomic nerve function in a state where device main body 10 is mounted on the wrist by mount 30. Therefore, since the user's both hands free even when determining the autonomic nerve function, the problems in daily living can be suppressed.

In addition, biological information measurement device 1 determines the cause of the deterioration of the autonomic nerve function based on the correlation coefficient between the sleeping time and sleeping time to be calculated by the body motion signal of body motion detector 24 and the degree of the autonomic nerve activity. Therefore, the user can confirm the cause of the deterioration of the autonomic nerve function.

In addition, biological information measurement device 1 provides the advice information base on the cause of the deterioration of the autonomic nerve function. Therefore, since the user suppress the deterioration of the autonomic nerve function based on the advice information, the daily living can be improved.

In addition, biological information measurement device 1 display the measurement results of the degree of the autonomic nerve activity, the number of steps, and the sleeping time, the cause of the deterioration of the autonomic nerve function, and the advice information on display 11. Therefore, the user can visually confirm the information displayed on display 11.

### Second Exemplary Embodiment

Next, a second exemplary embodiment of the invention will be described.

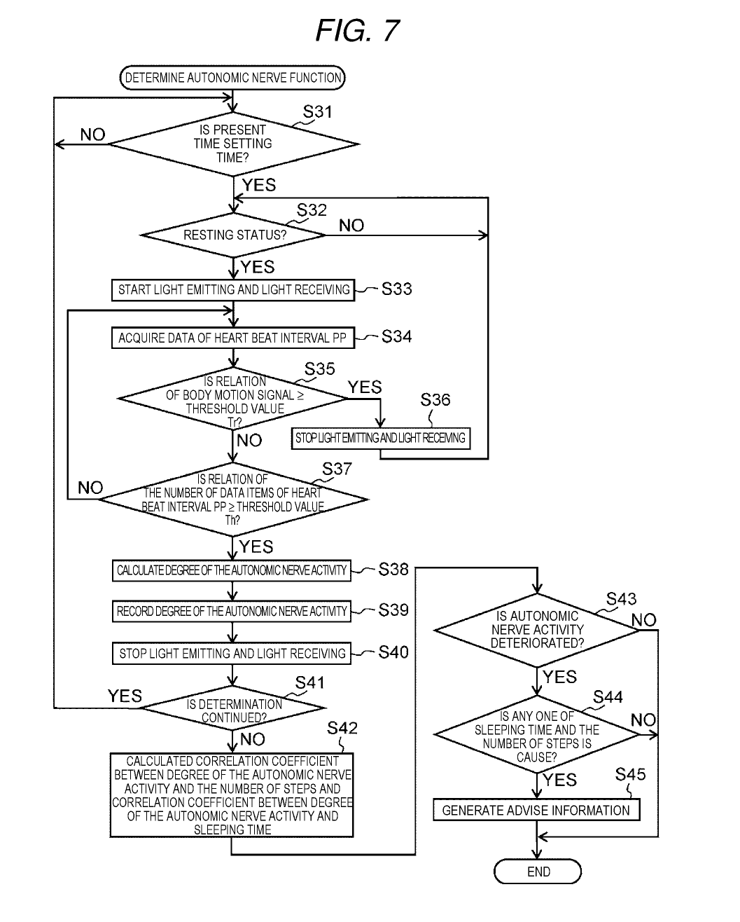

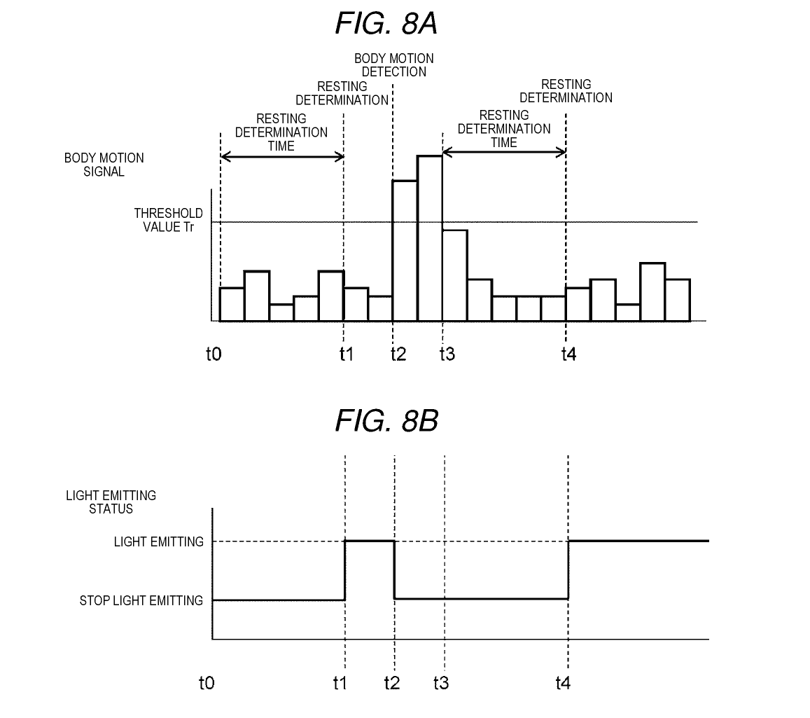

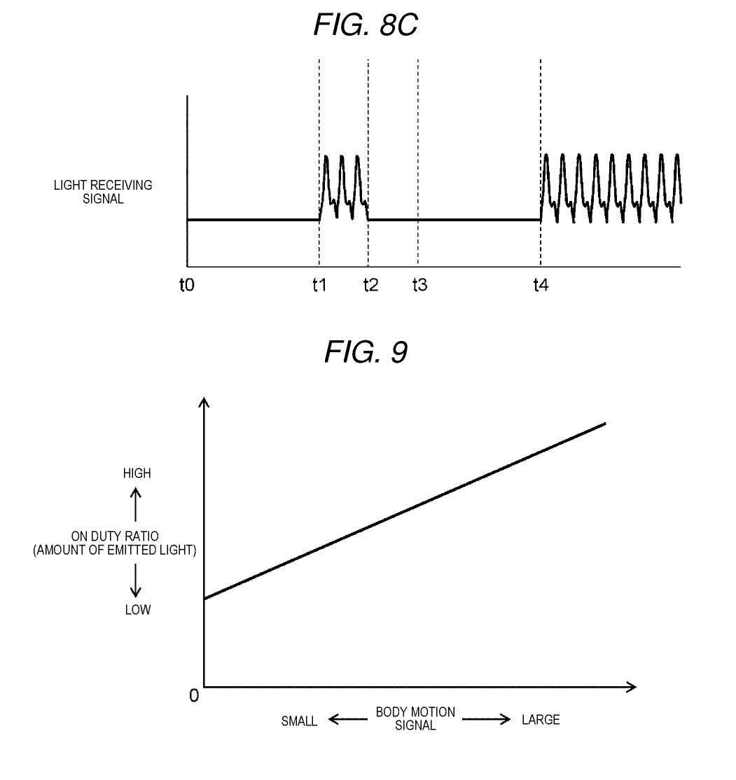

FIG. 7 is a flow chart illustrating a procedure of a determination of an autonomic nerve function of a second exemplary embodiment of the invention. FIG. 8A is a diagram illustrating a body motion signal of the second exemplary embodiment of the invention. FIG. 8B is a diagram illustrating a light emitting state of a light emitter of the second exemplary embodiment of the invention. FIG. 8C is a diagram illustrating a light receiving signal of the second exemplary embodiment of the invention.

Biological information measurement device 1 according to the second exemplary embodiment will be described with reference to FIGS. 7, 8A, and 8B. The procedure of the determination of the autonomic nerve function of biological information measurement device 1 according to the second exemplary embodiment and the procedure of the autonomic nerve function according to the first exemplary embodiment are different from each other in that the operation of light emitter 22 of pulse wave detector 21 is controlled based on the present time and the body motion signal and the light emitting of light emitter 22 is controlled based on the body motion signal.

Hereinafter, a feature different from biological information measurement device 1 according to the first exemplary embodiment will be described in detail. Same reference numerals are given to those components that are the same as the corresponding components of biological information measurement device 1 according to the first exemplary embodiment. Such components will not be described in detail. That is, in the flowing description, each configuration element of biological information measurement device 1 with reference numeral indicates each configuration element of biological information measurement device 1 of FIGS. 1 and 2.

When it is determined that the present time which is measured by time measuring instrument 28 is in a setting time that is a preset time, device main body 10 performs the determination of the autonomic nerve function. Therefore, controller 25 has the measuring mode and the sleeping mode as a control mode does not have the standby mode. The setting time is a time including a time when the determination of the autonomic nerve function starts and can set such that the user operates input component 12. As a starting time of the setting time, for example, times every 3 hours such as 8 o'clock, 11 o'clock, 14 o'clock, 17 o'clock, and 20 o'clock for a day are exemplified. The setting time is set as a minute from the predetermined time such as a time interval from 8:00 to 8:01, for example.

As described above, pulse wave detector 21 detects the pulse wave of the living body at the preset time.

In addition, when the body motion signal is equal to or more than threshold value Tr in the determination of the autonomic nerve function, device main body 10 causes light emitter 22 of pulse wave detector 21 to stop the light emitting and causes light receiver 23 of pulse wave detector 21 to stop the light receiving. After the light emitting of light emitter 22 and the light receiving of light receiver 23 are interrupted, when the body motion signal is less than threshold value Tr during the predetermined time (second predetermined time), device main body 10 determines whether the user in a resting status to causes the light emitter 22 to start the light emitting and light receiver 23 to start the light receiving again. The predetermined time is a resting determination time for determining whether the user is in the resting time and is set in advance by the examination or the like.

The procedure of the determination of the autonomic nerve function will be described using FIG. 7 with reference to FIGS. 1 and 2. In the flowing description, a portion of biological information measurement device 1 with a reference numeral indicates portions of biological information measurement devices 1 of FIGS. 1 and 2.

Controller 25 determines whether the present time is within the setting time (Step S31). When it is determined that the present time is not within the setting time (NO in Step S31), the process proceeds Step S31 again. On the other hand, when the present time is within the setting time (YES in Step S31), in Step S32, controller 25 determines whether the user is in the resting status.

When it is determined that the user is in the resting status (YES in Step S32), controller 25 causes light emitter 22 of pulse wave detector 21 to start light emitting and causes light receiver 23 of pulse wave detector 21 to start light receiving (Step S33). As an example, as shown in FIG. 8A, it is determined that time t0 that is a present time is 8 o'clock that is a setting time, the measurement of the resting determination time is started by timer 25b. In the resting determination time, it is determined whether the body motion signal is less than threshold value Tr. When the body motion signal is less than threshold value Tr from time t0 to time t1, that is, over the resting determination time, the light emitting of light emitter 22 and light receiving of light receiver 23 are started in time t1 as shown in FIGS. 8B and 8C. Therefore, the measurement of heart beat interval PP is started.

As described above, when light emitter 22 emits the light and light receiver 23 receives light, autonomic nerve index operator 26c acquires data of heart beat interval PP (Step S34). Autonomic nerve function determinator 26d determines whether the body motion signal is equal or more than threshold value Tr (Step S35).

When it is determined that the body motion signal is equal to or more than threshold value Tr (YES in Step S35), controller 25 determines that the body motion of the user is affected on the measurement of heart beat interval PP and influences of entropy value E caused by the body motion of increase. Controller 25 causes light emitter 22 to stop the light emitting and cause light receiver 23 to stop the light receiving (Step S36).

Therefore, the measurement of heart beat interval PP is interrupted. Next, the process proceeds to the determination of the resting status of Step S32. As an example, as shown in FIG. 8A, when the body motion signal is equal to or more than threshold value Tr in time t2, since the light emitting of light emitter 22 and light receiving of light receiver 23 are interrupted, the measurement of heart beat interval PP is interrupted. When the body motion signal is less than threshold value Tr in time t3, the measurement of the resting determination time is started. Since the body motion signal becomes less than threshold value Tr over the resting determination time that is times t3 to t4, the light emitting of light emitter 22 and light receiving of light receiver 23 in time t4 are started again.

On the other hand, when the body motion signal is not equal to or more than threshold value Tr in Step S35 (NO in Step S35), autonomic nerve function determinator 26d determines that the influences of entropy value E caused by the body motion is small. Therefore, in device main body 10, pulse wave detector 21 continues the measurement of heart beat interval PP. Autonomic nerve function determinator 26d determines whether the number of data items of heart beat interval PP is equal to or more than threshold value Th (Step S37). When it is determined that the number of data items of heart beat interval PP is not equal to or more than threshold value Th (NO in Step S37), the press proceeds to Step S34.

On the other hand, when it is determined that the number of data items of heart beat interval PP is equal to or more than threshold value Th (YES in Step S37), autonomic nerve index operator 26c calculates the degree of the autonomic nerve activity (Step S38) and records the degree of the autonomic nerve activity (Step S39). Controller 25 causes the light emitter 22 to stop the light emitting and the light receiver 23 to step the light receiving (Step S40).

Controller 25 determines whether or not to continue the determination of the autonomic nerve function (Step S41). In Step S41, all of the setting times which are identified from 0 o'clock to 24 o'clock for a day for example, controller 25 determines whether the calculation of the degree of the autonomic nerve activity is interrupted.

When it is determined that the determination is not to be continued (NO in Step S41), that is, when the calculation of the degree of the autonomic nerve activity in the all of the setting times for a day is terminated, the process proceeds Step S42. Since Steps S42 to S45 are same as Steps S15 to S18 of the determination of the autonomic nerve function according to the first exemplary embodiment, the description will be omitted.

On the other hand, when it is determine that the determination is to be continued (YES in Step S41), that is, when the calculation of the degree of the autonomic nerve activity in the all of the setting times for a day is not terminated, the presses proceeds to Step S31, and the calculation of the degree of the autonomic nerve activity is started again.

Biological information measurement device 1 has the following effects in addition to the effects of biological information measurement device 1 according to the first exemplary embodiment.

In the pulse wave information detected by pulse wave detector 21, a noise which causes the body motion of the living body is included. In the present exemplary embodiment, by controlling pulse wave detector 21 based on the body motion signal of the living body, it is possible to suppress that the body motion of the living body is affected on the detection of the pulse wave.

It is known that there is a daily fluctuation in the autonomic nerve function. Therefore, for reducing the influence of the daily fluctuation, in a case where the pulse wave of the living body is measured over a plurality number of days, the determination of the autonomic nerve function reduces is preferably performed at a predetermined time for a day. Biological information measurement device 1 performs the determination of the autonomic nerve function at a preset time based on the time which is measured by time measuring instrument 28. Therefore, in the determination of the autonomic nerve function, it is possible to suppress an influence of the daily fluctuation.

In addition, when the body motion signal of the living body is equal to or more than threshold value Tr, since the body motion of the living body exerts a large influence on the detection of the pulse wave, the detection of the pulse wave is interrupted by stopping the light emitting of the light emitter. Therefore, it is possible to suppress that the body motion of the living body is affected on the detection of the pulse wave.

In addition, when the body motion signal is equal to or more than threshold value Tr, since the light emitting of light emitter 22 and the light receiving of light receiver 23 are interrupted, entropy value E which is affected to the body motion is suppressed to be calculated. Therefore, data items of only the degree of the autonomic nerve activity when the user is in the resting status can be collected. Accordingly, the cause of the deterioration of the autonomic nerve function can be determined with good accuracy.

### Third Exemplary Embodiment

A third exemplary embodiment of the invention will be described.

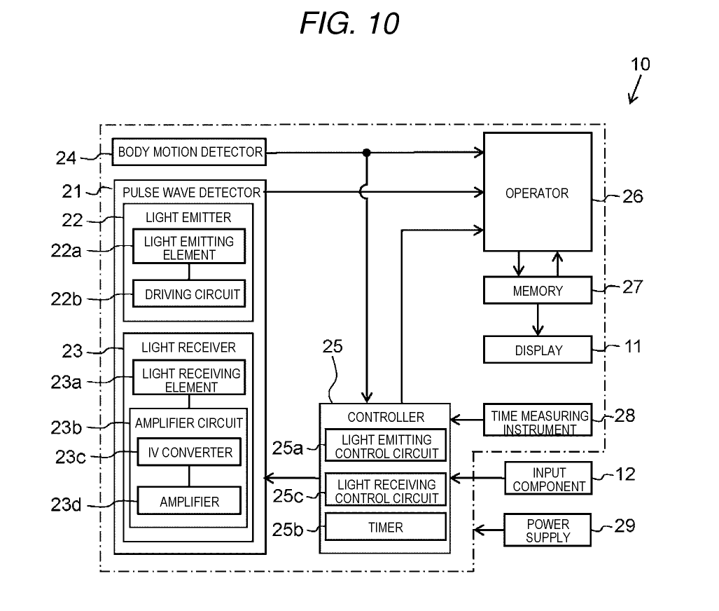

FIG. 9 is a diagram illustrating a relationship between an On DUTY ratio and a body motion signal of a driving circuit of the light emitter of a third exemplary embodiment of the invention.

In biological information measurement device 1 according to the third exemplary embodiment, controller 25 controls light emitter 22 of pulse wave detector 21 by a method different from that of biological information measurement device 1 according to the first exemplary embodiment. Hereinafter, a feature different from biological information measurement device 1 according to the first exemplary embodiment will be described in detail. Same reference numerals are given to those components that are the same as the corresponding components of biological information measurement device 1 according to the first exemplary embodiment. Such components will not be described in detail. That is, in the flowing description, a portion of biological information measurement device 1 with a reference numeral indicates portions of biological information measurement devices 1 of FIGS. 1 and 2.

Driving circuit 22b of light emitter 22 has a switch element (not shown). Driving circuit 22b drives a switch element by pulse width modulation (PWM) drive to cause light emitting element 22a to emit the light.

Controller 25 controls the amount of the emitted light of light emitter 22 of pulse wave detector 21 based on the body motion signal of body motion detector 24. The controlling is performed as follows.

A map illustrating the relationship between the body motion signal and an On DUTY ratio of driving circuit 22b of light emitter 22 as shown in FIG. 9 is stored in controller 25. The map of FIG. 9 illustrates a relationship where the On DUTY ratio of driving circuit 22b increases as the body motion signal becomes larger. Controller 25 generates the On DUTY ratio to be output from light emitting control circuit 25a to driving circuit 22b based on the map of FIG. 9. Therefore, since driving circuit 22b drives light emitter 22 based on the On DUTY ratio, the amount of emitted light of light emitter 22 is controlled based on the body motion signal.

An action of biological information measurement device 1 according to the exemplary embodiment will be described.

When the user moves its arm of which biological information measurement device 1 is mounted on the wrist, device main body 10 is displaced from the wrist. Accordingly, light of light emitter 22 leaks from between the wrist and device main body 10 in some cases. In addition, when user moves its arm, external light such as sunlight enters between the wrist and device main body 10 in some cases. Such as a leakage of the light and entering of the external light increases as the motion of the arm becomes greater, that is, the body motion signal become greater. Therefore, when the amount of emitted light of light emitter 22 is constant, since the amount of light radiated to the wrist decreases as the body motion signal becomes greater, the amount of the received light of light receiver 23 decreases.

The amount of the external light entering between the wrist and device main body 10 increases as the body motion signal becomes greater. Therefore, the noise included in the light receiving signal increases based on the reflected light from the wrist of light receiver 23. Therefore, it is difficult to obtain the received signal in accordance with the amount of the received light, by operator 26 with good accuracy. As a result, since it is difficult to detect the maximum value of the light receiving signal with good accuracy, the accuracy of the measurement of heart beat interval PP may be deteriorated.

Biological information measurement device 1 according to the present exemplary embodiment increases the On DUTY ratio of driving circuit 22b as the body motion signal becomes greater. Therefore, since the amount of the emitted light of light emitter 22 increases as the body motion signal becomes greater, it is suppress that the amount of the received light of light receiver 23 decreases when the arm moves furiously. Therefore, the reduction of the light receiving signal is suppressed. In addition, in a case where the noise is included in the light receiving signal by the external light, since the reduction of the light receiving signal is suppressed, the rate of the noise with respect to the light receiving signal becomes smaller. Accordingly, since the deterioration of the detection accuracy of the maximum value of the light receiving signal is suppressed, the reduction of the measurement accuracy of heart beat interval PP is suppressed.

Biological information measurement device 1 of the present exemplary embodiment has the following effects in addition to the effects of biological information measurement device 1 according to the first exemplary embodiment.

In biological information measurement device 1, since the amount of the emitted light of light emitter 22 increases as the body motion signal becomes greater, the reduction of the measurement accuracy of heart beat interval PP is suppressed. Therefore, since the reduction of the accuracy of the calculation value of the degree of the autonomic nerve activity based on heart beat interval PP is suppressed, the reduction of the determination accuracy of the autonomic nerve function is suppressed. Accordingly, even when the user exercising for example, the determination of the autonomic nerve function can be performed.

In addition, when the body motion signal is small, since the On DUTY ratio of driving circuit 22b is reduced, the amount of the emitted light of light emitter 22 can be suppressed. Accordingly, it is possible to reduce power consumption of power supply 29.

### Fourth Exemplary Embodiment

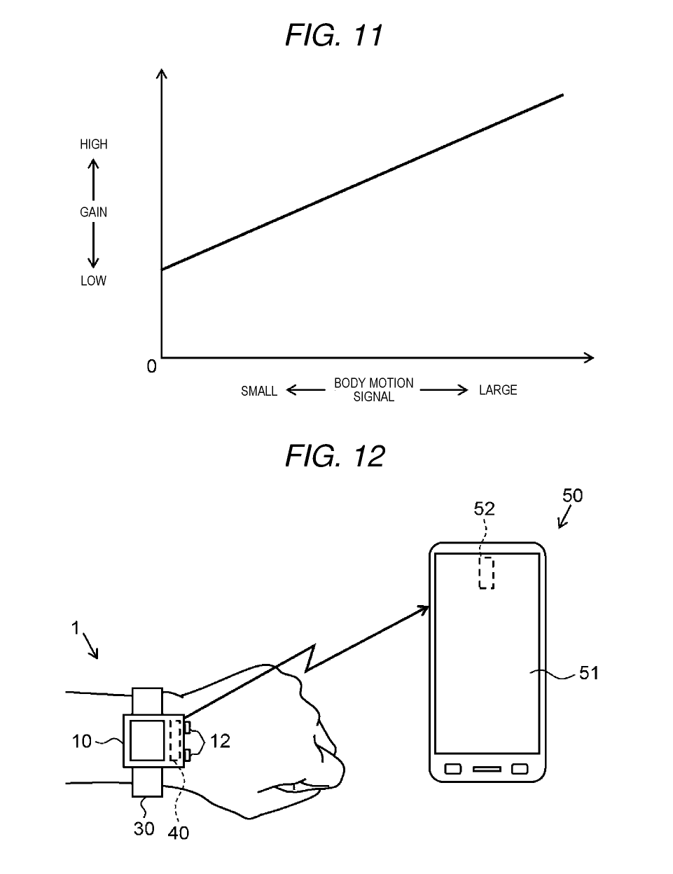

FIG. 10 is a block diagram illustrating a control configuration of biological information measurement device 1 of a fourth exemplary embodiment of the invention. In addition, FIG. 11 is a diagram illustrating a relationship between a gain and a body motion signal of a light receiver of the fourth exemplary embodiment of the invention.

In biological information measurement device 1 according to the fourth exemplary embodiment, controller 25 controls light receiver 23 of pulse wave detector 21 by a method different from biological information measurement device 1 according to the first exemplary embodiment. Hereinafter, features different from biological information measurement device 1 according to the first exemplary embodiment will be described in detail. Same reference numerals are given to those components that are the same as the corresponding components of biological information measurement device 1 according to the first exemplary embodiment. Such components will not be described in detail.

As shown in FIG. 10, light receiver 23 includes amplifier circuit 23b for amplifying the signal of light receiving element 23a. Amplifier circuit 23b includes IV converter 23c for converting an electric current signal to be generated by light receiving element 23a based on the reflected light to a voltage signal and amplifier 23d for amplifying a voltage output by IV converter 23c. Amplifier 23d changes a gain which is a ratio of voltage signal Vin input to amplifier 23d and voltage signal Vout output from amplifier 23d. Voltage signal Vout output from amplifier 23d is output to operator 26 as a light receiving signal. Therefore, the light receiving signal increases as the gain of amplifier 23d becomes greater.

Controller 25 includes light receiving control circuit 25c for adjusting the grain of amplifier circuit 23b. The a map illustrating the relationship between the body motion signal and the gain of amplifier 23d is stored in controller 25 as shown in FIG. 11. The map illustrates that the gain of amplifier 23d increases as the body motion signal becomes greater. Controller 25 sets the gain of amplifier 23d from the body motion signal using the map of FIG. 11. Light receiving control circuit 25c adjusts the gain of amplifier circuit 23b so as to be a gain set by the map of FIG. 11.

Biological information measurement device 1 according to the present exemplary embodiment has the following effects in addition to the effects of biological information measurement device 1 according to the first exemplary embodiment.

As described in the third exemplary embodiment, since the amount of the received light of light receiver 23 as the body motion signal becomes greater, the light receiving signal is decreased. Biological information measurement device 1 of the present exemplary embodiment, the gain of amplifier 23d is increased as the body motion signal becomes greater. Therefore, the reduction of the light receiving signal caused by increasing the gain of amplifier 23d as the amount of the received light becomes smaller, and the increase in the ratio of the noise with respect to the light receiving signal are suppressed. Accordingly, the deterioration of the measurement accuracy of heart beat interval PP is suppressed. Accordingly, since the deterioration of the accuracy of the calculation value of the degree of the autonomic nerve activity based on heart beat interval PP is suppressed, the reduction of the accuracy of the determination of the autonomic nerve function is suppressed.

### Fifth Exemplary Embodiment

A fifth exemplary embodiment of the invention will be described.

FIG. 12 is a configuration diagram illustrating a configuration of biological information measurement device 1 of a fifth exemplary embodiment of the invention.

Biological information measurement device 1 according to the fifth exemplary embodiment and biological information measurement device 1 according to the first exemplary embodiment are different to each other in that biological information measurement device 1 is communicated with portable information terminal 50 which is an example of the external instrument. Hereinafter, a feature different from biological information measurement device 1 according to the first exemplary embodiment will be described in detail. Same reference numerals are given to those components that are the same as the corresponding components of biological information measurement device 1 according to the first exemplary embodiment. Such components will not be described in detail. That is, in the following description, each portion of biological information measurement device 1 with a reference numeral indicates each portion of biological information measurement device 1 of FIGS. 1 and 2.

Device main body 10 of biological information measurement device 1 includes communicator 40 which communicates with portable information terminal 50 that is the external instrument in a wireless manner. Controller 25 of device main body 10 includes a communicating mode communicating with portable information terminal 50. By operating input component 12 by the user, controller 25 sets the operating mode to the communicating mode. When controller 25 is in the communicating mode, biological information measurement device 1 becomes in a state where biological information measurement device 1 can communicate with portable information terminal 50.

An example of portable information terminal 50 is a smartphone. Portable information terminal 50 includes communicator 52 which communicates with biological information measurement device 1 in a wireless manner. Portable information terminal 50 and biological information measurement device 1 are communicated with each other by using Bluetooth (registered trademark) which is an example of a communication method.

Portable information terminal 50 displays the number of steps, the sleeping time, the degree of the autonomic nerve activity, and the determination result of the autonomic nerve function which are communicated from biological information measurement device 1 on display 51. A display area of display 51 is larger than a display area of display 11 of device main body 10. As a detailed example of display 51, graphs in FIGS. 3A to 3C, graphs in FIGS. 4A and 4B, the display of the advice information, and the like are included. That is, for displaying the determination result of the autonomic nerve function on the external instrument, communicator 40 communicates the determination result of the autonomic nerve function to communicator 52 of the external instrument.

Biological information measurement device 1 according to the present exemplary embodiment has the following effects in addition to biological information measurement device 1 according to the first exemplary embodiment.

Since the information of biological information measurement device 1 is displayed on display 51 of portable information terminal 50, each information item can be largely displayed as compared to a case where the information of biological information measurement device 1 is displayed on display 11 of device main body 10. Accordingly, the user easily confirms the information such as the determination result of the autonomic nerve function or the like via portable information terminal 50.

### Sixth Exemplary Embodiment

A sixth exemplary embodiment of the invention will be described.

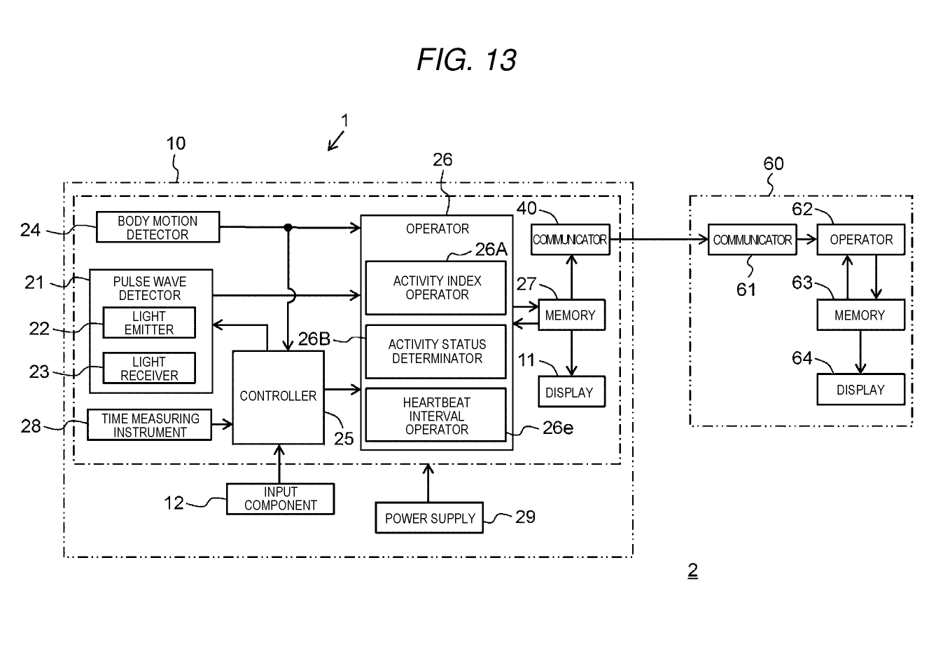

FIG. 13 is a block diagram illustrating a control configuration of a biological information measurement system of a sixth exemplary embodiment of the invention.

Biological information measurement system 2 according to the sixth exemplary embodiment includes biological information measurement device 1 and external instrument 60. Biological information measurement device 1 according to the present exemplary embodiment has the different control configuration as compared with the control configuration of biological information measurement device 1 according to the fifth exemplary embodiment. Hereinafter, a feature different from biological information measurement device 1 according to the first exemplary embodiment will be described in detail. In addition, same reference numerals are given to those components that are the same as the corresponding components of biological information measurement device 1 according to the first exemplary embodiment. Such components will not be described in detail.

Operator 26 of device main body 10 of biological information measurement device 1 does not have autonomic nerve index operator 26c and autonomic nerve function determinator 26d. On the other hand, operator 26 includes heartbeat interval operator 26e for calculating heart beat interval PP. Heartbeat interval operator 26e outputs calculated heart beat interval PP to memory 27.

An example of external instrument 60 is a desktop personal computer (PC). External instrument 60 includes communicator 61, operator 62, memory 63, and display 64. Communicator 61 communicates heart beat interval PP calculated by operator 26 and the activity status (the number of steps and the sleeping time) based on the body motion signal between communicator 61 and communicator 40. Operator 62 and memory 63 correspond to operator 26 and memory 27 of biological information measurement device 1 according to the first exemplary embodiment. External instrument 60 performs the determination of the autonomic nerve function.

The operation of the determination of the autonomic nerve function according to biological information measurement system 2 will be described.

When controller 25 is set to the measuring mode by operating of input component 12 by the user, light emitting of light emitter 22 and light receiving of light receiver 23 are started. Memory 27 stores heart beat interval PP calculated based on the light receiving signal and the number of steps and the sleeping time calculated based on the body motion signal of body motion detector 24.

When heart beat interval PP, the number of steps, and the sleeping time acquired in biological information measurement device 1 are communicated to external instrument 60, the user sets controller 25 to the communicating mode by operating of input component 12. Therefore, heart beat interval PP, the number of steps, and the sleeping time are communicated to external instrument 60 from biological information measurement device 1.

External instrument 60 performs the determination of the autonomic nerve function based on heart beat interval PP, the number of steps, and the sleeping time, in the same manner as the first exemplary embodiment. External instrument 60 stores the determination result of the autonomic nerve function. External instrument 60 displays the determination result of the autonomic nerve function by the operation of the user on display 64.

Biological information measurement system 2 has the following effects in addition to the effects of biological information measurement device 1 according to the fifth exemplary embodiment.

The user communicates heart beat interval PP, the number of steps, and the sleeping time to external instrument 60. Accordingly, external instrument 60 determines the autonomic nerve function. Therefore, the electrocardiogram monitor for detecting the pulse wave and the control device for determining the autonomic nerve function may not be provided in biological information measurement device 1, like the conventional autonomic nerve function evaluation device. Therefore, the user can carry biological information measurement device 1. If the user communicates heart beat interval PP, the number of steps, and the sleeping time stored in device main body 10 without carrying external instrument 60, external instrument 60 can determine the autonomic nerve function. Therefore, in biological information measurement system 2, the user may carry only biological information measurement device 1. Accordingly, since the user's both hands free even when determining the autonomic nerve function, the problems in daily living can be suppressed.

### Modification Exemplary Embodiment

Next, a modification example of the invention will be described.

The detailed aspect to be obtained by the present biological information measurement device and the present biological information measurement system is not limited to the aspects illustrated the each exemplary embodiment. In the range where the object of the invention can be achieved, the present biological information measurement device and the present biological information measurement system can obtain an aspect different from the aspects according to the first exemplary embodiment to the sixth exemplary embodiment. A modification example to be described below of the exemplary embodiments is an example of various aspects which can be obtained by the present biological information measurement device and the present biological information measurement system.

In biological information measurement device 1 according to the first exemplary embodiment, the detection of the pulse wave may be always performed by pulse wave detector 21. In this case, controller 25 omit the standby mode.

In biological information measurement device 1 according to the second exemplary embodiment, the preset time may be a time per day in the determination of the autonomic nerve function.

Device main body 10 of the second exemplary embodiment may include a notifying unit. When the present time becomes the setting time, the notifying unit informs the user that the pulse wave is measured by pulse wave detector 21 with sound or buzzer sound. In this case, in the determination of the autonomic nerve function, instead of the determination that it is in the resting status or not by the user, the notifying unit informs that it is in the resting status or not.

In device main body 10 according to the second exemplary embodiment, as a setting time, the setting time may be a setting time for each predetermined time during the sleeping time. When the user is in the bedtime, since the high probability of being resting status, it is possible to acquire data of heart beat interval PP stably.

After the degree of the daily autonomic nerve activity is recorded, the device main body 10 according to the second exemplary embodiment may perform the determination of the degree of the autonomic nerve function after single recording the degree of the autonomic nerve activity instead of the determination of the autonomic nerve function. In this case, the process of the determination whether the determination of the autonomic nerve function of Step S41 is continued proceeds after generating of the advice information of Step S45.

Biological information measurement devices 1 according to the fifth exemplary embodiment and the sixth exemplary embodiment may not be provide display 11.

Portable information terminal 50 according to the fifth exemplary embodiment may be a tablet type information terminal or a laptop PC instead of the smartphone. The external instrument including portable information terminal 50 is not limited to a portable type, and may be a desktop PC or other stationary information terminals.

Portable information terminal 50 according to the fifth exemplary embodiment display only one of the activity status of the number of steps and the sleeping time, and the determination result of the autonomic nerve function. In a case where only determination of the autonomic nerve function is displayed, portable information terminal 50 may not display the cause of the deterioration of the autonomic nerve function.

External instrument 60 according to the sixth exemplary embodiment may be portable information terminal 50 instead of the desktop PC. In this case, portable information terminal 50 has operator 62 and memory 63. In addition, external instrument 60 may be the stationary information, for example, a dedicated information terminal for performing the determination of the autonomic nerve function instead of the PC.

Light emitting elements 22a according to the first exemplary embodiment and the sixth exemplary embodiment may be a green light emitting diode or a light emitting diode including a plurality of wavelengths of red light, green light, or the light.

Light emitting elements 22a according to the first exemplary embodiment to the sixth exemplary embodiment may have a plurality of light emitting diodes.

Body motion detectors 24 according to the first exemplary embodiment to the sixth exemplary embodiment may have an angular velocity sensor or an air pressure sensor instead of an acceleration sensor.

Memories 27 according to the first exemplary embodiment to the sixth exemplary embodiment may be a hard disk, a flash memory, or a solid state drive (SSD) instead of a RAM.

Activity index operators 26a according to the first exemplary embodiment to the sixth exemplary embodiment may calculate at least one of a sleep depth and a sleep efficiency as the activity index, instead of the sleeping time.

Activity index operators 26a according to according to the first exemplary embodiment to the sixth exemplary embodiment may calculate at least one of metabolic equivalents (METs) and a consumed calorie, as the activity status.

Device main body 10 according to the first exemplary embodiment to the fifth exemplary embodiment and external instrument 60 according to the sixth exemplary embodiment may be use the METs and the consumed calorie for determination of the cause of the deterioration of the autonomic nerve function based on the relationship between the METs and the consumed calorie, and the degree of the autonomic nerve activity.

As a calculation of entropy value E, autonomic nerve index operators 26c according to the first exemplary embodiment to the sixth exemplary embodiment may use a method for calculating a Renyi entropy instead of a method for calculating by the average information amount of Shannon shown in the (Formula 2) expression.

Autonomic nerve index operators 26c according to the first exemplary embodiment to the sixth exemplary embodiment may calculate an index of a balance of the autonomic nerve function (balance between a function of a sympathetic nerve and a function of a parasympathetic nerve) as an autonomic nerve index. The index of the balance of the autonomic nerve function can be calculated by a tone value of a tone entropy method, or a ratio (LF/HF) of a low frequency (LF) component and a high frequency (HF) component of a frequency analysis method.

As an autonomic nerve index, autonomic nerve index operators 26c according to the first exemplary embodiment to the sixth exemplary embodiment may calculate total power of the frequency analysis method instead of entropy value E.

Autonomic nerve function determinators 26d according to the first exemplary embodiment to the sixth exemplary embodiment may be performed a total determination based on the sleeping time, the sleeping efficiency, the number of steps, and the consumed calorie as the activity index, the degree of the autonomic nerve activity as the autonomic nerve index, and the autonomic nerve balance. As an example, autonomic nerve function determinator 26d may have a configuration for evaluating the sleeping time, the sleeping efficiency, the number of steps, the consumed calorie, the degree of the autonomic nerve activity, and the autonomic nerve balance with 5 grades, and generating a radar chart.

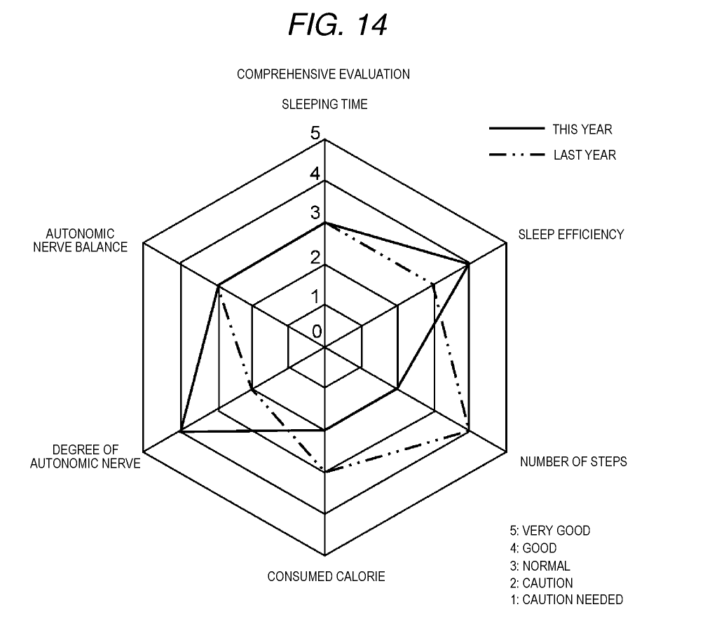

FIG. 14 is a radar chart illustrating a comprehensive evaluation which is obtained by using the biological information measurement device of a modification example of the invention.

In FIG. 14, a comprehensive evaluation of this year (solid line) and a comprehensive evaluation of the last year (two-dot chain line) are compared and displayed. By calculating the area surrounded by a solid line that is the comprehensive evaluation of this year and the area surrounded by the two-dot chain line that is the comprehensive evaluation, autonomic nerve function determinator 26d calculate comprehensive determination values of this year and last year. The comprehensive determination values indicate that the comprehensive evaluation is good as the area becomes larger. By comparing the area of the comprehensive evaluation in this year and the area of the comprehensive evaluation in last year, autonomic nerve function determinator 26d determines whether the comprehensive evaluation of this year of is improved than the evaluation of the last year.

In the radar chart of FIG. 14, autonomic nerve function determinator 26d may delete items of a part of the sleeping time, the sleeping efficiency, the number of steps, and the consumed calorie, the degree of the autonomic nerve activity, and the autonomic nerve balance in a range in which the radar chart can generate.

Autonomic nerve function determinators 26d according to the first exemplary embodiment to the sixth exemplary embodiment may change threshold value Ts of the correlation coefficient between the number of steps and the degree of the autonomic nerve activity and the correlation coefficient between the sleeping time and the degree of the autonomic nerve activity based on at least one of gender of age. For example, a table of threshold value Ts for each of the gender and the age may be stored in memory 27. The user inputs the gender and the age through the operating input component 12. Therefore, threshold value Ts is set in accordance with the input gender and the age.

Autonomic nerve function determinators 26d according to the first exemplary embodiment to the sixth exemplary embodiment may determine a cause of the deterioration of the autonomic nerve function based on an index obtained by other statistical techniques of a first main component using a main component analysis, a discriminant function using a discriminant analysis, or the like instead of the correlation coefficient as an index.

Autonomic nerve function determinators 26d according to the first exemplary embodiment to the sixth exemplary embodiment may determine the cause of the deterioration of the autonomic nerve function based on the fact that the calculated index is greater than a determination standard, for example, when the average value of the past indexes such as the correlation coefficient or a standard deviation is used as the determination standard.

Device main bodies 10 according to the first exemplary embodiment to the sixth exemplary embodiment may include a signal processor for removing a noise of the light receiving signal based on the body motion signal of body motion detector 24. In the light receiving signal, a body motion signal component is contained as the noise. The signal processor removes the body motion signal component from the light receiving signal. Therefore, heart beat interval PP can be measured with high accuracy.

In the measurement of the sleeping time, device main bodies 10 according to the first exemplary embodiment to the fifth exemplary embodiment and external instrument 60 according to the sixth exemplary embodiment may perform a determination whether the accumulated value of the body motion signal is equal to or more than the threshold value at a certain time, instead of the determination whether the body motion signal is equal to or more than threshold value Ts. In this case, when the accumulated value is equal to or more than the threshold value at a certain time, controller 25 stops the light emitting of the light emitter 22. When the accumulated value is less than the threshold value at a certain time, controller 25 acquires data of heart beat interval PP.

In the measurement of the sleeping time, device main bodies 10 according to the first exemplary embodiment to the fifth exemplary embodiment and external instrument 60 according to the sixth exemplary embodiment may determine as compared with the body motion signal using the threshold value of the value different from threshold value Tr of the body motion signal.

In the determination of the autonomic nerve function, biological information measurement devices 1 according to first exemplary embodiment to the fifth exemplary embodiment and external instrument 60 according to the sixth exemplary embodiment may not perform the determination of the type of the activity status which causes the deterioration of the autonomic nerve function.

Biological information measurement devices 1 according to first exemplary embodiment to the sixth exemplary embodiment may not include mount 30. In this case, for example, when biological information measurement device 1 is stored in user's cloth pocket, the user can carry biological information measurement device 1. In this case, when determining the autonomic nerve function, the user places biological information measurement device 1 on the wrist thereof. In this case, the user may maintain biological information measurement device 1 so as to fix biological information measurement device 1 on the wrist by a user's hand.

Pulse wave detector 21 according to the first exemplary embodiment may adopt at least one method of a method of controlling light emitter 22 according to the second exemplary embodiment, and a method of controlling light emitter 22 according to the third exemplary embodiment.

Pulse wave detector 21 according to the second exemplary embodiment may adopt a method of controlling light emitter 22 according to the third exemplary embodiment.

Pulse wave detector 21 according to the third exemplary embodiment may adopt a method of controlling light receiver 23 according to the fourth exemplary embodiment.

The fifth exemplary embodiment may adopt biological information measurement devices 1 according to the second exemplary embodiment to the fourth exemplary embodiment instead of biological information measurement device 1 according to the first exemplary embodiment.

Biological information measurement device 1 according to the sixth exemplary embodiment may adopt a control configuration of biological information measurement devices 1 according to the first exemplary embodiment to the fourth exemplary embodiment. In this case, the determination of the autonomic nerve function may be performed by any one of biological information measurement device 1 and portable information terminal 50.

(An Example of Aspect which can be Obtained by Biological Information Measurement Device and Biological Information Measurement System)

(1) According to an independent aspect of the present biological information measurement device, the biological information measurement device includes a device main body including a pulse wave detector having a light emitter for irradiating a living body with light and a light receiver for receiving reflected light when the light emitter irradiate the living body with the light, and detecting pulse wave of the living body based on the reflected light received in the light receiver. The device main body determines an autonomic nerve function based on the pulse wave information of the living body detected by the pulse wave detector.

According to the biological information measurement device, the device main body includes an optical system pulse wave detector, and determines the autonomic nerve function. Therefore, the electrocardiogram monitor for detecting the pulse wave and the control device for determining the autonomic nerve function are not necessarily formed separate from the device main body, like the conventional autonomic nerve function evaluation device. Therefore, the user can carry the biological information measurement device.

(2) According to an aspect of the biological information measurement device, the main body includes a body motion detector for detecting a body motion of the living body and may have a configuration for controlling the pulse wave detector based on a body motion signal indicating the body motion information of the living body detected by the body motion detector.

In the pulse wave information detected by the pulse wave detector, an noise which causes the body motion of the living body is included. According to the biological information measurement device, by controlling the pulse wave detector based on the body motion information of the living body, it is possible to suppress that the body motion of the living body is affected on the detection of the pulse wave.

(3) According to an aspect of the biological information measurement device, the device main body may stop the light emitting of the light emitter when the body motion of the living body detected by the body motion detector is equal to or more than the threshold value.

According to the biological information measurement device, when the body motion of the living body is equal to or more than the threshold value, since the body motion of the living body exerts a large influence on the detection of the pulse wave, the device main body stops the light emitting of the light emitter to spot the detection of the pulse wave. Therefore, it is possible to suppress that the body motion of the living body is affected on the detection of the pulse wave.

(4) According to an aspect of the biological information measurement device, the device main body may have a configuration for increasing an amount of the emitted light of the light emitter as the body motion of the living body detected by the body motion detector becomes greater.

According to the biological information measurement device, for example, when the body motion of the living body flutters during measuring of the pulse wave, since the device main body is displaced with respect to the living body, a case where light of the light emitter is leak between the device main body and the living body occurs. In this case, the amount of the emitted light of the light emitter is increased. Therefore, it is possible to suppress deterioration in a detection accuracy of the pulse wave of the living body. In addition, when the body motion of the living body during measuring of the pulse wave not flutter or the living body is not be moved, the device main body is not easily displaced with respect to the living body. Therefore, in a case where the light of the light emitter is not leak between the device main body and the living body, the amount of the emitted light of the light emitter is decreased. Therefore, it is possible to reduce power consumption of the device main body.

(5) According to an aspect of the biological information measurement device, the light receiver includes an amplifier circuit for amplifying a signal in accordance with the reflected light received, and the amplifier circuit may have a configuration for increasing signals as the body motion of the living body detected by the body motion detector flutters.

According to the biological information measurement device, for example, when the body motion of the living body flutters, since the device main body is displaced with respect to the living body, a case where light of the light emitter is leak between the device main body and the living body occurs. In this case, the signal in accordance with the amount of the received light is increased by the amplifier circuit of the light receiver. Therefore, it is possible to suppress the deterioration of the detection accuracy of the pulse wave of the living body due to a decrease in the signal in accordance with the amount of the received light.

(6) According to an aspect of the biological information measurement device, the device main body may have a configuration for detecting the pulse wave of the living body by the pulse wave detector at a preset time.

According to the biological information measurement device, in a case where the pulse wave of the living body is measured for the number of days, since the pulse wave of the living body is detected every days at the same time, it is possible to reduce the influence of daily fluctuation.

(7) According to an aspect of the biological information measurement device, the device main body includes a body motion detector for detection the body motion of the living body and the pulse wave information of the living body detected by the pulse wave detector may have a configuration for determining an autonomic nerve function based on the body motion information of the living body detected by the body motion detector.

(8) According to an aspect of the biological information measurement device, the device main body may have a configuration for measuring the activity status based on the body motion information of the living body detected by the body motion detector and measuring the type of the activity status which causes the deterioration of the autonomic nerve function based on the activity status and the autonomic nerve function.

According to the biological information measurement device, from the activity status and the autonomic nerve function based on the body motion information of the living body detected by the body motion detector, the relationship between the autonomic nerve function and the activity status can be found. Therefore, when the autonomic nerve function is deteriorated, it is possible to determine that the activity status having a high relationship between the autonomic nerve function and thereof is a cause of deterioration in the autonomic nerve function.

(9) According to an aspect of the biological information measurement device, the device main body may have a configuration for providing information for alleviating the deterioration of the autonomic nerve function, based on the determination result of the type of the activity status which causes deterioration of the autonomic nerve function.

According to the biological information measurement device, when the user obtains the information for alleviating the deterioration of the autonomic nerve function, the daily life can be improved based on the information. Therefore, the user can efficiently alleviate the deterioration of the autonomic nerve function.

(10) According to an aspect of the biological information measurement device, the device main body includes a communicator which communicates with the external instrument. In order that the determination result of the autonomic nerve function is displayed on the external instrument, the device main body may have a configuration which communicates the determination result of autonomic nerve function to the external instrument by the communicator.

(11) According to an independent aspect of the present biological information measurement system, there is provided a biological information measurement system including a biological information measurement device having a device main body including a pulse wave detector having a light emitter for irradiating a living body with light and a light receiver for receiving reflected light when the light emitter irradiates the living body with the light, and detecting pulse wave information of the living body based on the reflected light received in the light receiver; and an external instrument which can communicate with the biological information measurement device. The device main body includes a communicator which communicates pulse wave information of the living body detected by the pulse wave detector to the external instrument. The external instrument may have a configuration for determining an autonomic nerve function based on the pulse wave of the living body which is communicated by the communicator.

According to the biological information measurement system, when user communicates the pulse wave information of the living body to the external instrument, the external instrument determines the autonomic nerve function. Therefore, the biological information measurement device may not include the electrocardiogram monitor for detecting the pulse wave and the control device for determining the autonomic nerve function like the autonomic nerve function evaluation device. Therefore, the user can carry the biological information measurement device. Even when the user does not carry the external instrument, if when communicating the body motion information of the living body and the body pulse information of the living body stored in the device main body, the external instrument can determine the autonomic nerve function. Therefore, in the biological information measurement system, the user may carry only biological information measurement device.

### INDUSTRIAL APPLICABILITY

As described above, since the biological information measurement device and the biological information measurement system according to the invention can demonstrate outstanding effects that the biological information measurement device and the biological information measurement system can be carried by the user, the biological information measurement device and the biological information measurement system are suitable for use as a device and a system for diagnosing the autonomic nerve function.

### REFERENCE MARKS IN THE DRAWINGS

1 biological information measurement device
2 biological information measurement system
10 device main body
10a device main body case
10b surface
10c rear surface
10d side surface
11 display
12 input component
21 pulse wave detector
22 light emitter
22a light emitting element
22b driving circuit
23 light receiver
23a light receiving element
23b amplifier circuit
23c IV converter
23d amplifier
24 body motion detector
25 controller
25a light emitting control circuit
25b timer
25c light receiving control circuit
26 operator
26a activity index operator
26b activity status determinator
26c autonomic nerve index operator
26d autonomic nerve function determinator
26e heartbeat interval operator
27 memory
28 time measuring instrument
29 power supply
30 mount
40 communicator
50 portable information terminal
51 display
52 communicator
60 external instrument
61 communicator
62 operator
63 memory
64 display

1. A biological information measurement device comprising:
a pulse wave detector having a light emitter for irradiating a living body with light and a light receiver for receiving reflected light of the light emitted from the living body when the light emitter irradiates the living body with the light, the pulse wave detector detecting pulse wave information of the living body based on the reflected light received in the light receiver;
an operator for determining an autonomic nerve function based on the pulse wave information of the living body detected by the pulse wave detector;
a controller configured so as to control the pulse wave detector and the operator; and
a measuring device main body case provided with the pulse wave detector, the operator, and the controller.

2. The biological information measurement device according to claim 1, further comprising:
a body motion detector for detecting a body motion of the living body,
wherein the controller is configured so as to control the pulse wave detector based on a body motion signal indicating the body motion of the living body detected by the body motion detector.

3. The biological information measurement device according to claim 2,
wherein the controller is configured so as to cause the light emitter to stop emitting the light, when the body motion signal detected by the body motion detector is equal to or more than a predetermined threshold value.

4. The biological information measurement device according to claim 2,
wherein the controller is configured so as to increase an amount of emitted light of the light emitter as the body motion signal detected by the body motion detector becomes greater.

5. The biological information measurement device according to claim 2,
wherein the light receiver has an amplifier circuit for amplifying an input signal in accordance with the received reflected light and outputting an output signal, and
the controller is configured so as to set a gain of the amplifier circuit such that the amplifier circuit increases the output signal as the body motion of the living body detected by the body motion detector becomes greater.

6. The biological information measurement device according to claim 1,
wherein the pulse wave detector detects a pulse wave of the living body at a preset time.

7. The biological information measurement device according to claim 1, further comprising:
a body motion detector for detecting body motion information of the living body,
wherein the autonomic nerve function is determined based on the pulse wave information of the living body detected by the pulse wave detector and the body motion information of the living body detected by the body motion detector.

8. The biological information measurement device according to claim 7,
wherein the operator measures an activity status based on the body motion information of the living body and determines a type of the activity status which causes deterioration of the autonomic nerve function based on the activity status and the autonomic nerve function.

9. The biological information measurement device according to claim 8,
wherein the controller is configured so as to provide information for alleviating deterioration of the autonomic nerve function based on a determination result of the type of the activity status which causes deterioration of the autonomic nerve function.

10. The biological information measurement device according to claim 7, further comprising:
a communicator configured to communicate with an external instrument,
wherein the communicator communicates a determination result of the autonomic nerve function to the external instrument to display the determination result on the external instrument.

11. The biological information measurement device according to claim 1, further comprising:
a display provided in the device main body case and configured so as to display the determination result of the autonomic nerve function; and
a mount configured so as to mount the device main body case to a wrist of a user.

12. The biological information measurement device according to claim 8,
wherein the operator includes an autonomic nervous index operator and an autonomic nervous determinator,
the autonomic nervous index operator calculates a degree of autonomic nervous activity that is an autonomic nervous index, and
the autonomic nervous determinator determines whether the type of the activity status which causes the deterioration of the autonomic nerve function is the number of steps or a sleeping time, based on a correlation coefficient between the degree of the autonomic nerve activity and the number of steps and the sleeping time to be calculated by the body motion signal detected by the body motion detector.

13. The biological information measurement device according to claim 3,
wherein after the controller stops light emitting of the light emitter, the controller is configured so as to restart light emitting of the light emitter and to restart light receiving of the light receiver, when the body motion signal is less than the predetermined threshold value for a predetermined time.

14. The biological information measurement device according to claim 4,
wherein the light emitter includes a light emitting element and a driving circuit for driving the light emitting element, and
the controller increases an On DUTY ratio of the driving circuit as the body motion of the living body detected by the body motion detector becomes greater, and increases an amount of emitted light of the light emitting element.

15. A biological information measurement system comprising:
a biological information measurement device including a pulse wave detector having a light emitter for irradiating a living body with light and a light receiver for receiving reflected light of the light emitted from the living body when the light emitter irradiates the living body with the light, and detecting pulse wave information of the living body based on the reflected light received in the light receiver; and
an external instrument which can communicate with the biological information measurement device,
wherein the biological information measurement device includes a communicator which communicates the pulse wave information of the living body detected by the pulse wave detector to the external instrument, and
the external instrument determines an autonomic nerve function based on the pulse wave information of the living body communicated by the communicator.

16. The biological information measurement device according to claim 3,
wherein the controller is configured so as to increase an amount of emitted light of the light emitter as the body motion signal detected by the body motion detector becomes greater.

17. The biological information measurement device according to claim 16,
wherein the light emitter includes a light emitting element and a driving circuit for driving the light emitting element, and
the controller increases an On DUTY ratio of the driving circuit as the body motion of the living body detected by the body motion detector becomes greater, and increases an amount of emitted light of the light emitting element.
